(19) United States
(12) Patent Application Publication
(10) Pub. No.: US 2026/0256612 A1
(43) Pub. Date: Sep. 3, 2026

(54) FLUID COLLECTION ASSEMBLIES INCLUDING AT LEAST ONE SHAPE MEMORY MATERIAL DISPOSED IN THE CONDUIT

(71) Applicant: PureWick Corporation, Covington, GA (US)

(72) Inventors: Andrew Meyer, Oyster Bay, NY (US); Benjamin Jackson, El Cajon, CA (US); Sarah Skelton, Springboro, OH (US); Nicholas Austerman, Atlanta, GA (US); Christopher K. Brooks, Decatur, GA (US); Morgan Rex, El Cajon, CA (US); Cassie Singleton, El Cajon, CA (US); Benjamin Myers, Atlanta, GA (US); Alexis Nunn, Illijay, GA (US)

(21) Appl. No.: 19/541,655

(22) Filed: Feb. 17, 2026

Related U.S. Application Data
(63) Continuation of application No. 17/451,354, filed on Oct. 19, 2021, now Pat. No. 12,569,365.
(60) Provisional application No. 63/094,626, filed on Oct. 21, 2020.

(51) Int. Cl. A61F 5/455; A61F 5/44; B29C 48/16

(52) U.S. Cl. CPC A61F 5/455; A61F 5/4404; B29C 48/16

(57) ABSTRACT

Embodiments disclosed herein are related to assemblies, systems, and methods of using fluid collection assemblies and systems. The assemblies, systems, and methods of using fluid collection assemblies and systems include at least one shape memory material for forming and maintaining the fluid collection assembly into a selected shape. The shape memory material may enable for selective manipulation of the fluid collection assembly to contour to the anatomical features of variously sized patients.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection assemblies continue to seek new and improved assemblies, systems, and methods to collect urine.

### SUMMARY

Embodiments disclosed herein are related to assemblies, systems, and methods of using fluid collection assemblies and systems. In an embodiment, a fluid collection assembly is disclosed. The fluid collection assembly includes a fluid impermeable barrier at least partially defining a chamber, at least one opening configured to be positioned adjacent to a female urethral opening, and a fluid outlet. The fluid collection assembly also includes at least one porous material disposed in the chamber. Further, the fluid collection assembly includes a conduit extending from the fluid outlet into the chamber, the conduit including one or more walls that define a passageway. Additionally, the fluid collection assembly includes at least one shape memory material at least partially disposed within the one or more walls of the conduit. The at least one shape memory material is sized, shaped, and positioned in the one or more walls to retain a selected geometric configuration.

In an embodiment, a system is disclosed. The system includes a fluid collection assembly. The fluid collection assembly includes a fluid impermeable barrier at least partially defining a chamber, at least one opening configured to be positioned adjacent to a female urethral opening, and a fluid outlet. The fluid collection assembly also includes at least one porous material disposed in the chamber. Further, the fluid collection assembly includes a conduit extending from the fluid outlet into the chamber, the conduit including one or more walls that define a passageway. Additionally, the fluid collection assembly includes at least one shape memory material at least partially disposed within the one or more walls of the conduit. The at least one shape memory material is sized, shaped, and positioned to retain a selected geometric configuration. The system also includes a fluid storage container configured to hold a fluid and a vacuum source fluidly coupled to one or more of the fluid storage container or the fluid collection assembly via the conduit. The vacuum source is configured to remove fluid from the fluid collection assembly via the conduit.

In an embodiment, a method of forming a fluid collection assembly is disclosed. The method includes providing a conduit and at least one shape memory material, the conduit includes one or more walls that define a passageway, the at least one shape memory material at least partially disposed in the one or more walls. The at least one shape memory material is sized, shaped, and positioned to retain a selected geometric configuration. The method also includes positioning at least a portion the conduit through a fluid outlet and into a chamber defined by a fluid impermeable barrier. The fluid impermeable barrier defines at least one opening configured to be positioned adjacent to a female urethral opening. The method also includes positioning at least one porous material in the chamber.

In an embodiment, a method to collect one or more bodily fluids is disclosed. The method includes positioning an opening of a fluid collection assembly adjacent to a female urethral opening. The fluid collection assembly includes a fluid impermeable barrier at least partially defining a chamber, at least one opening configured to be positioned adjacent to a female urethral opening, and a fluid outlet. The fluid collection assembly also includes at least one porous material disposed in the chamber. Further, the fluid collection assembly includes a conduit extending from the fluid outlet into the chamber, the conduit including one or more walls that define a passageway. Additionally, the fluid collection assembly includes at least one shape memory material at least partially disposed within the one or more walls of the conduit. The at least one shape memory material is sized, shaped, and positioned to retain a selected geometric configuration. The method also includes shaping the fluid collection assembly into the selected geometric configuration. The selected geometric configuration is complementary to contours of anatomy of a patient in a region proximate to the female urethral opening. The method further includes receiving fluid from the female urethral opening into a chamber of the fluid collection assembly.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIGS. 1A and 1B are isometric views of a fluid collection assembly exhibiting a first shape and a second shape, respectively, according to an embodiment.

FIGS. 1C and 1D are cross-sectional schematics of the fluid collection assembly shown in FIG. 1B taken along planes 1C-1C and 1D-1D, respectively.

FIG. 2A is a cross-sectional schematic of a conduit that includes a plurality of shape memory materials disposed therein, according to an embodiment.

FIG. 2B is a cross-sectional schematic of the conduit taken along plane 2B-2B shown in FIG. 2A.

FIG. 3 is an isometric view of a portion of a conduit that includes at least one shape memory material that does not extend generally parallel to a longitudinal axis of the conduit, according to an embodiment.

FIG. 4A is an isometric view of a portion of a conduit that includes at least one shape memory material, according to an embodiment.

FIG. 4B is an isometric view of a portion of a conduit that includes a plurality of shape memory materials at least partially disposed therein, according to an embodiment.

FIG. 5 is a cross-sectional schematic of a portion of a conduit that includes at least one shape memory material disposed therein, according to an embodiment.

FIG. 6A is an isometric view of a portion of a conduit that includes at least one shape memory material at least partially disposed therein, according to an embodiment.

FIG. 6B is an isometric view of a portion of a conduit that includes at least one shape memory material at least partially disposed therein, according to an embodiment.

FIG. 6C is an cross-sectional schematic of a portion of a conduit that includes at least one shape memory material at least partially disposed therein, according to an embodiment.

FIG. 7A is a cross-sectional schematic of a fluid collection assembly, according to an embodiment.

FIG. 7B is a cross-sectional schematic of the fluid collection assembly taken along plane 7B-7B of FIG. 7A.

FIG. 8 is a block diagram of a system for fluid collection, according to an embodiment.

FIG. 9 is flow diagram of a method to form any of the fluid collection assemblies disclosed herein.

FIG. 10 is a flow diagram of a method to collect one or more bodily fluids, according to an embodiment.

### DETAILED DESCRIPTION

Embodiments disclosed herein are related to assemblies, systems, and methods of using fluid collection assemblies and systems. The assemblies, systems, and methods of using fluid collection assemblies and systems include at least one shape memory material for forming and maintaining the fluid collection assembly into a selected shape. The shape memory material may enable for selective manipulation of the fluid collection assembly to contour to the anatomical features of variously sized patients.

The fluid collection assemblies disclosed herein include a fluid impermeable barrier. The fluid impermeable barrier at least defines a chamber, at least one opening extending therethrough that is configured to be positioned adjacent to a female urethral opening, and a fluid outlet. The fluid collection assemblies disclosed herein may include at least one porous material disposed in the chamber and a conduit at least partially disposed in the chamber (e.g., extending through the fluid outlet). The fluid collection assemblies disclosed herein include at least one shape memory material at least partially disposed in the conduit.

During use, the female fluid collection assembly is disposed with the opening of the fluid collection assembly adjacent to a urethral opening of a female patient. The urethral opening may discharge one or more bodily fluids, such a urine, blood, or sweat. The bodily fluids may be received by the porous material and wicked away from the urethral opening and into the chamber. The bodily fluids may be removed from the chamber via a fluid outlet defined by the fluid impermeable barrier. For example, a suction force may be applied to a conduit that is at least partially disposed in the fluid outlet and the suction force may pull the bodily fluids into the conduit and out of the chamber.

Some of the bodily fluids that are discharged from the urethral opening may leak from the chamber. The leaked bodily fluids may cause patient (e.g., an individual using the fluid collection assembly) discomfort, embarrassment, and create unsanitary conditions that require cleaning. Poor fit between the female fluid collection assembly and the region about the urethral opening may cause the bodily fluids to leak from the female fluid collection assembly. For example, the poor fit may cause gaps to be present between the porous material that extends across the opening and the region about the urethral opening. These gaps may provide locations through which the bodily fluids may flow without being received by the porous material and/or locations at which bodily fluids may leave the porous material. To minimize formation of gaps between the porous material and the urethral opening, conventional fluid collection assemblies (e.g., fluid collection assemblies without a shape memory material) may be manipulated (e.g., bent or otherwise shaped) to match the anatomical shape of the patient to conform to the shape of the vaginal region of the patient. Conventional fluid collection assemblies rely on contact between the thighs of the patient and the conventional fluid collection assemblies to maintain the shape of the conventional fluid collection assemblies. However, movement by the patient, thin patients, young patients, and forgetful patients (e.g., patients with dementia) may have issues maintaining contact between the thighs and the conventional fluid collection assemblies. As such, movement by the patient, thin patients, young patients, and forgetful patients may cause the conventional fluid collection assemblies to change a shape thereof which, in turn, may form gaps through which the bodily fluids may leak.

The shape of the fluid collection assemblies disclosed herein may be manipulated into a selected shape to provide a more comfortable and effective fit on the patient. The shape memory material of the fluid collection assemblies disclosed herein may at least temporarily maintain the fluid collection assemblies in a selected shape. By shaping and maintaining the shape of the fluid collection assembly with the shape memory material to match the anatomical shape of the patient, more of the fluid may be collected and retained in the fluid collection assembly. For example, shaping the fluid collection assembly to match the anatomical shape of the patient inhibits the fluid collection assembly from moving away from the groin of the patient (e.g., when the patient moves). Moving the fluid collection assembly away from the patient increases the likelihood that the bodily fluids leak from the fluid collection assembly during use. As such, shaping the fluid collection assembly with the shape memory material to match the anatomical shape of the wear minimizes leaks.

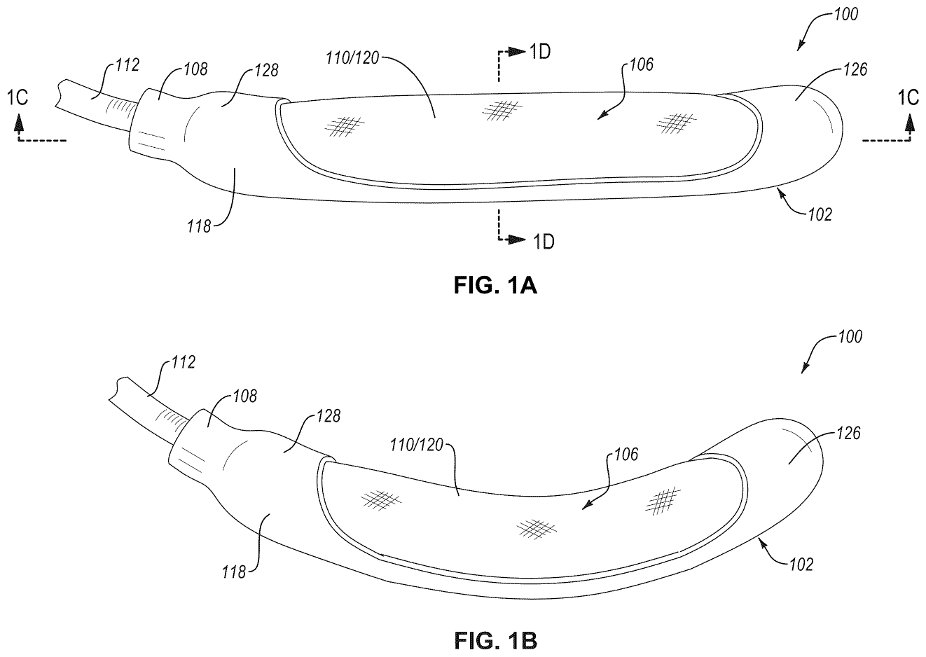

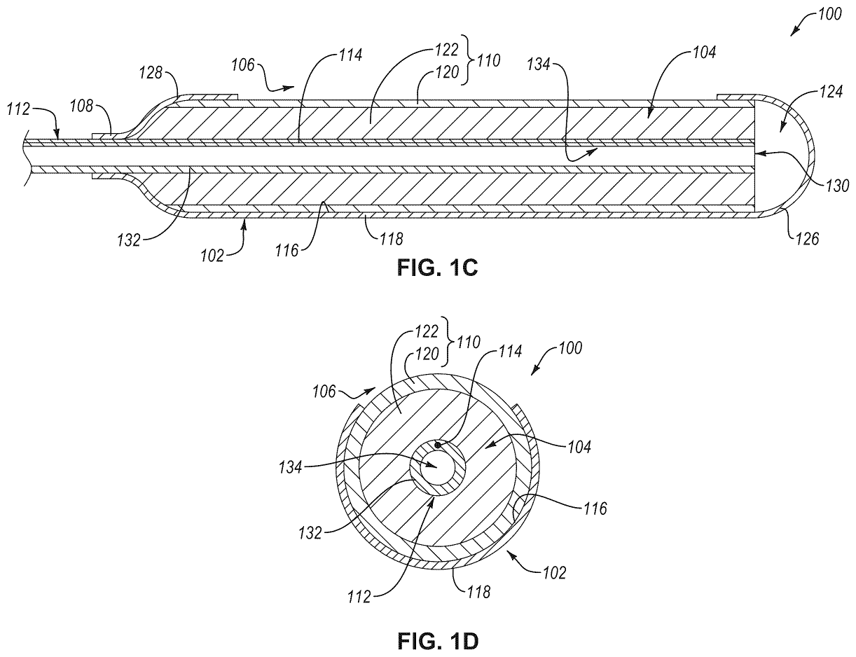

FIGS. 1A and 1B are isometric views of a fluid collection assembly 100 exhibiting a first shape and a second shape, respectively, according to an embodiment. FIGS. 1C and 1D are cross-sectional schematics of the fluid collection assembly 100 shown in FIG. 1B taken along planes 1C-1C and 1D-1D, respectively. The fluid collection assembly 100 is an example of a female fluid collection assembly 100 for receiving and collection bodily fluids from a female patient. The fluid collection assembly 100 includes a fluid impermeable barrier 102 that defines at least a chamber 104, at least one opening 106, and a fluid outlet 108. The fluid collection assembly 100 also includes at least one porous material 110 (e.g., wicking material) disposed in the chamber 104. The fluid collection assembly 100 further includes at least one conduit 112 at least partially disposed within the chamber 104 and at least one shape memory material 114 at least partially disposed within the conduit 112.

The fluid impermeable barrier 102 at least partially defines a chamber 104 (e.g., interior region) and an opening 106. For example, the interior surface(s) 116 of the fluid impermeable barrier 102 at least partially defines the chamber 104 within the fluid collection assembly 100. The fluid impermeable barrier 102 temporarily stores bodily fluids in the chamber 104. The fluid impermeable barrier 102 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface 118 of the fluid impermeable barrier 102 may be formed from a soft and/or smooth material, thereby reducing chaffing.

In some examples, the fluid impermeable barrier 102 may be tubular (ignoring the opening), such as substantially cylindrical (as shown), oblong, prismatic, or flattened tubes. During use, the outer surface 118 of the fluid impermeable barrier 102 may contact the patient. The fluid impermeable barrier 102 may be sized and shaped to fit in the gluteal cleft between the legs of a female patient.

The opening 106 provides an ingress route for fluids to enter the chamber 104. The opening 106 may be defined by the fluid impermeable barrier 102 such as by an inner edge of the fluid impermeable barrier 102. For example, the opening 106 is formed in and extends through the fluid impermeable barrier 102, from the outer surface 118 to the inner surface 116, thereby enabling the bodily fluids to enter the chamber 104 from outside of the fluid collection assembly 100. The opening 106 may be an elongated hole in the fluid impermeable barrier 102. For example, the opening 106 may be defined as a cut-out in the fluid impermeable barrier 102. The opening 106 may be located and shaped to be positioned adjacent to a female urethral opening.

The fluid collection assembly 100 may be positioned proximate to the female urethral opening and urine may enter the chamber of the fluid collection assembly 100 via the opening 106. The fluid collection assembly 100 is configured to receive the bodily fluids into the chamber 104 via the opening 106. When in use, the opening 106 may have an elongated shape that extends from a first location below the urethral opening (e.g., at or near the anus or the vaginal opening) to a second location above the urethral opening (e.g., at or near the top of the vaginal opening or the mons pubis).

The opening 106 may have an elongated shape because the space between the legs of a female is relatively small when the legs of the female are closed, thereby only permitting the flow of the bodily fluids along a path that corresponds to the elongated shape of the opening 106 (e.g., longitudinally extending opening). The opening 106 in the fluid impermeable barrier 102 may exhibit a length that is measured along the longitudinal axis of the fluid collection assembly 100 that may be at least about 10% of the length of the fluid collection assembly 100, such as about 25% to about 50%, about 40% to about 60%, about 50% to about 75%, about 65% to about 85%, or about 75% to about 95% of the length of the fluid collection assembly 100.

The opening 106 in the fluid impermeable barrier 102 may exhibit a width that is measured transverse to the longitudinal axis of the fluid collection assembly 100 that may be at least about 10% of the circumference of the fluid collection assembly 100, such as about 25% to about 50%, about 40% to about 60%, about 50% to about 75%, about 65% to about 85%, or about 75% to about 100% of the circumference of the fluid collection assembly 100. The opening 106 may exhibit a width that is greater than 50% of the circumference of the fluid collection assembly 100 since the vacuum (e.g., suction) through the conduit 112 pulls the fluid through the porous material 110 and into the conduit 112. In some examples, the opening 106 may be vertically oriented (e.g., having a major axis parallel to the longitudinal axis of the assembly 100). In some examples (not shown), the opening 106 may be horizontally oriented (e.g., having a major axis perpendicular to the longitudinal axis of the assembly 100). In an example, the fluid impermeable barrier 102 may be configured to be attached to the patient, such as adhesively attached (e.g., with a hydrogel adhesive) to the patient. According to an example, a suitable adhesive is a hydrogel layer.

In some examples, the fluid impermeable barrier 102 may define an fluid outlet 108 sized to receive the conduit 112. The at least one conduit 112 may be disposed in the chamber 104 via the fluid outlet 108. The fluid outlet 108 may be sized and shaped to form an at least substantially fluid tight seal against the conduit 112 or the at least one tube, thereby substantially preventing the bodily fluids from escaping the chamber 104.

The fluid impermeable barrier 102 may include markings (not shown) thereon, such as one or more markings to aid a patient in aligning the assembly 100 on the patient. For example, a line on the fluid impermeable barrier 102 (e.g., opposite the opening 106) may allow a healthcare professional to align the opening 106 over the urethral opening of the patient. In examples, the markings may include one or more of alignment guide or an orientation indicator, such as a stripe or hashes. Such markings may be positioned to align the assembly 100 to one or more anatomical features such as a pubic bone, etc.

The fluid collection assembly 100 includes porous material 110 disposed in the chamber 104. The porous material 110 may cover at least a portion (e.g., all) of the opening 106. The porous material 110 is exposed to the environment outside of the chamber 104 through the opening 106. The porous material 110 may be configured to move any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” Such “wicking” may not include absorption of fluid into the porous material. Put another way, substantially no absorption of fluid into the material may take place after the material is exposed to the fluid and removed from the fluid for a time. While no absorption is desired, the term “substantially no absorption” may allow for nominal amounts of absorption of fluid into the porous material (e.g., absorbency), such as less than about 30 wt % of the dry weight of the porous material, less than about 20 wt %, less than about 15 wt %, less than about 10 wt %, less than about 7 wt %, less than about 5 wt %, less than about 3 wt %, less than about 2 wt %, less than about 1 wt %, or less than about 0.5 wt % of the dry weight of the porous material. The porous material 110 may also wick the fluid generally towards an interior of the chamber 104, as discussed in more detail below. The porous material 110 may include one or more of a fluid permeable membrane 120 or a fluid permeable support 122. However, in some embodiments, it is noted that the porous material 110 may include an absorption material (e.g., hydrophilic material) instead of a wicking material.

The fluid collection assembly 100 may include the fluid permeable membrane 120 disposed in the chamber 104. The fluid permeable membrane 120 may cover at least a portion (e.g., all) of the opening 106. The fluid permeable membrane 120 may be composed to wick fluid away from the opening 106, thereby preventing the fluid from escaping the chamber 104.

The fluid permeable membrane 120 may include any material that may wick the fluid. For example, the fluid permeable membrane 120 may include fabric, such as a gauze (e.g., a silk, linen, or cotton gauze), another soft fabric, or another smooth fabric. Forming the fluid permeable membrane 120 from gauze, soft fabric, and/or smooth fabric may reduce chaffing caused by the fluid collection assembly 100.

The fluid collection assembly 100 may include the fluid permeable support 122 disposed in the chamber 104. The fluid permeable support 122 is configured to support the fluid permeable membrane 120 since the fluid permeable membrane 120 may be formed from a relatively foldable, flimsy, or otherwise easily deformable material. For example, the fluid permeable support 122 may be positioned such that the fluid permeable membrane 120 is disposed between the fluid permeable support 122 and the fluid impermeable barrier 102. As such, the fluid permeable support 122 may support and maintain the position of the fluid permeable membrane 120. The fluid permeable support 122 may include any material that may wick the fluid, such as any of the fluid permeable membrane materials disclosed herein above. For example, the fluid permeable membrane material(s) may be utilized in a more dense or rigid form than in the fluid permeable membrane 120 when used as the fluid permeable support 122. The fluid permeable support 122 may be formed from any fluid permeable material that is less deformable than the fluid permeable membrane 120. For example, the fluid permeable support 122 may include a porous polymer (e.g., nylon, polyester, polyurethane, polyethylene, polypropylene, etc.) structure or an open cell foam, such as spun nylon fibers. In some examples, the fluid permeable support 122 may be formed from a natural material, such as cotton, wool, silk, or combinations thereof. In such examples, the material may have a coating to prevent or limit absorption of fluid into the material, such as a water repellent coating. In some examples, the fluid permeable support 122 may be formed from fabric, felt, gauze, or combinations thereof. In some examples, the fluid permeable membrane 120 may be optional. For example, the porous material 110 may include only the fluid permeable support 122. In some examples, the fluid permeable support 122 may be optionally omitted from the fluid collection assembly 100. For example, the porous material 110 may only include the fluid permeable membrane 120.

The fluid permeable support 122 may have a greater ability to wick fluids than the fluid permeable membrane 120, such as to move the fluid inwardly from the outer surface of the fluid collection assembly 100. In some examples, the wicking ability of the fluid permeable support 122 and the fluid permeable membrane 120 may be substantially the same.

The fluid permeable membrane 120 and the fluid permeable support 122 may at least substantially completely fill the portions of the chamber 104 that are not occupied by the conduit 112. In some examples, the fluid permeable membrane 120 and the fluid permeable support 122 may not substantially completely fill the portions of the chamber 104 that are not occupied by the conduit 112. In such an example, the fluid collection assembly 100 includes the fluid reservoir 124 (FIG. 1C) disposed in the chamber 104.

The fluid reservoir 124 is a substantially unoccupied portion of the chamber 104. The fluid reservoir 124 may be defined between the fluid impermeable barrier 102 and one or both of the fluid permeable membrane 120 and fluid permeable support 122. The bodily fluids that are in the chamber 104 may flow through the fluid permeable membrane 120 and/or fluid permeable support 122 to the fluid reservoir 124. The fluid reservoir 124 may retain of the bodily fluids therein.

The bodily fluids that are in the chamber 104 may flow through the fluid permeable membrane 120 and/or fluid permeable support 122 to the fluid reservoir 124. The fluid impermeable barrier 102 may retain the bodily fluids in the fluid reservoir 124. While depicted at the distal end region 126, the fluid reservoir 124 may be located in any portion of the chamber 104 such as the proximal end region 128. The fluid reservoir 124 may be located in a portion of the chamber 104 that is designed to be located in a gravimetrically low point of the fluid collection assembly when the assembly is worn.

In some examples (not shown), the fluid collection assembly 100 may include multiple reservoirs, such as a first reservoir that is located at the portion of the chamber 104 closest to the inlet 130 (e.g., distal end region 126) and a second reservoir that is located at the portion of the of the chamber 104 that is closest to the outlet of the conduit 112 (e.g., proximal end region 128). In another example, the fluid permeable support 122 is spaced from at least a portion of the conduit, and the fluid reservoir 124 may be the space between the fluid permeable support 122 and the conduit.

The conduit 112 includes one or more walls 132 defining a passageway 134. The conduit 112 may be at least partially disposed in the chamber 104. The conduit 112 may be used to remove bodily fluids from the chamber 104. The conduit 112 (e.g., a tube) includes an inlet 130 and an outlet positioned downstream from the inlet 130. The outlet may be operably coupled to a suction source, such as a vacuum pump for withdrawing fluid form the chamber through the conduit 112. For example, the conduit 112 may extend into the fluid impermeable barrier 102 from the proximal end region 128 and may extend to the distal end region 126 to a point proximate to the fluid reservoir 124 therein such that the inlet 130 is in fluid communication with the fluid reservoir 124. The conduit 112 fluidly couples the chamber 104 with the fluid storage container (not shown) or the vacuum source (not shown).

The conduit 112 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 112 may include silicon or latex. In some examples, the conduit 112 may include one or more portions that are resilient, such as to by having one or more of a diameter or wall thickness that allows the conduit to be flexible.

As shown in FIG. 1C, the end of the conduit 112 may extend through a bore in the fluid permeable membrane 120 and/or fluid permeable support 122, such as into the fluid reservoir 124. For example, the inlet 130 may be extend into or be positioned in the fluid reservoir 124. In the illustrated embodiment, the conduit 112 is at least partially disposed in the fluid reservoir 124. In some examples (not shown), the conduit 112 may enter the chamber 104 in the distal end region 126 and the inlet 130 of the conduit 112 may be disposed in the distal end region 126 (e.g., in the fluid reservoir 124). The fluid collected in the fluid collection assembly 100 may be removed from the chamber 104 via the conduit 112.

In some examples, the inlet 130 may not extend into the fluid reservoir 124. In such examples, the inlet 130 may be disposed within the porous material 110 (fluid permeable membrane 120 and/or fluid permeable support 122) or at a terminal end thereof. For example, an end of the conduit 112 may be coextensive with or recessed within the fluid permeable membrane 120 and/or fluid permeable support 122.

Locating the inlet 130 at or near a location expected to be the gravimetrically low point of the chamber 104 when worn by a patient enables the conduit 112 to receive more of the bodily fluids than if the inlet 130 was located elsewhere and reduces the likelihood of pooling (e.g., pooling of the bodily fluids may cause microbe growth and foul odors). For instance, the bodily fluids in the fluid permeable membrane 120 and the fluid permeable support 122 may flow in any direction due to capillary forces. However, the bodily fluids may exhibit a preference to flow in the direction of gravity, especially when at least a portion of the fluid permeable membrane 120 and/or the fluid permeable support 122 is saturated with the bodily fluids. Accordingly, one or more of the inlet 130 or the fluid reservoir 124 may be located in the fluid collection assembly in a position expected to be the gravimetrically low point in the fluid collection assembly when worn by a patient, such as the distal end region 126.

In an example, the conduit 112 is configured to be at least insertable into the chamber 104. In such an example, the conduit 112 may include one or more markers (not shown) on an exterior thereof that are located to facilitate insertion of the conduit 112 into the chamber 104. For example, the conduit 112 may include one or more markings thereon that are configured to prevent over or under insertion of the conduit 112, such as when the conduit 112 defines an inlet 130 that is configured to be disposed in or adjacent to the fluid reservoir 124. In another example, the conduit 112 may include one or more markings thereon that are configured to facilitate correct rotation of the conduit 112 relative to the chamber 104. The one or more markings may include a line, a dot, a sticker, or any other suitable marking.

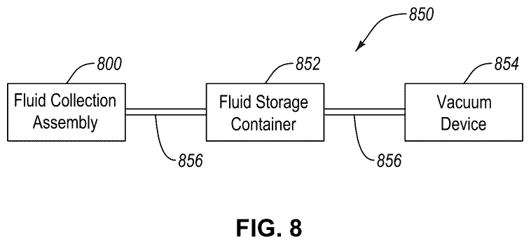

As described in more detail below, the conduit 112 is configured to be coupled to, and at least partially extend between, one or more of the fluid storage container (not shown) and the vacuum source (FIG. 8). In an example, the conduit 112 is configured to be directly connected to the vacuum source. In such an example, the conduit 112 may extend from the fluid impermeable barrier 102 by at least one foot, at least two feet, at least three feet, or at least six feet. In another example, the conduit 112 is configured to be indirectly connected to at least one of the fluid storage container (FIG. 8) and the vacuum source. In some examples, the conduit is secured to a patient's skin with a catheter securement assembly, such as a STATLOCK® catheter securement assembly available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 130 and the outlet 112 are configured to fluidly couple (e.g., directly or indirectly) the vacuum source (not shown) to the chamber 104 (e.g., the fluid reservoir 124). As the vacuum source (FIG. 8) applies a vacuum/suction in the conduit 112, the bodily fluids in the chamber 104 (e.g., at the distal end region 126, such as in the fluid reservoir 124) may be drawn into the inlet 130 and out of the fluid collection assembly 100 via the conduit 112. In some examples, the conduit may be frosted or opaque (e.g., black) to obscure visibility of the bodily fluids therein.

The fluid collection assembly 100 includes a shape memory material 114 at least partially disposed in the walls 132 of the conduit 112. The shape memory material may be sized, shaped, and positioned in the fluid collection assembly 100 to cause at least a portion of the fluid collection assembly 100 to retain a selected shape (e.g., geometric configuration). In an embodiment, the shape memory material 114 is configured to be bent, shaped, or otherwise deformed (hereafter collectively referred to as “shape,” “shaped,” or “shaping”). In an example, the shape memory material 114 is configured to be shaped along an entire length thereof. Allowing the shape memory material 114 to be shaped along the entire length thereof may allow the fluid collection assembly 100 to exhibit a shape that substantially corresponds to the anatomical features of the patient. For example, the shape memory material 114 may exhibit a first (e.g., initial) shape. The fluid collection assembly 100 may exhibit the first configuration (i.e., a generally linear shape shape) illustrated in FIG. 1A when the shape memory material 114 exhibits the first shape (e.g., a generally straight shape). The shape memory material 114 may be shaped to exhibit a second shape that is different than the first shape. The fluid collection assembly 100 may exhibit the second configuration (e.g., a generally curved shape) illustrated in FIG. 1B when the shape memory material 114 exhibits the second shape. The second configuration of the fluid collection assembly 100 may better correspond to the shape of the region about the urethral opening than the first configuration.

In an example, the shape memory material 114 is configured to be shaped at one or more selected location thereof. In such an example, the selected locations of the shape memory material 114 may be preferentially shaped relative to the rest of the shape memory material 114. While configuring the shape memory material 114 to be shaped at the selected location may inhibit the fluid collection assembly 100 from exhibiting a shape that substantially corresponds to the anatomical features of the patient, it may facilitate shaping of the fluid collection assembly 100, especially for less experienced medical individuals (e.g., medical practitioners or patients). In an embodiment, the shape memory material 114 may not be configured to be shaped. Instead, the shape memory material may exhibit a selected shape that corresponds or substantially corresponds to the anatomical feature of the patient. In such an embodiment, the shape memory material 114 may be more rigid and/or resilient than the rest of the fluid collection assembly 100 thereby causing at least a portion of the fluid collection assembly 100 to correspond to the selected shape of the shape memory material 114.

The shape memory material 114 may include a shape memory polymer or a metal (e.g., shape memory metal). Generally, the shape memory material 114 is composed to adopt an intermediate or permanent shape in response to a stimuli. For example, the shape memory material 114 may exhibit a first (e.g., initial) shape and may be switched from the first shape to a second shape by the stimuli, wherein the second shape is different than the first shape. The shape memory material 114 may also be switched from the second shape back to the first shape or a third shape that is different than the first and second shapes in response to the stimuli.

The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials disclosed herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal wire bent to a first shape may be utilized as the shape memory material 114, whereinafter the metal wire may be modified to a second shape via physical force applied thereto or via heating. However, in some embodiments, the shape memory material 114 may exhibit a selected shape, as discussed above and application of the stimuli may cause the shape memory material to deform (e.g., elastically deform or bend) into an intermediate shape. In such embodiments, the shape memory material 114 may return to the first initial shape upon removal of the stimuli such that the shape memory material 114 does not maintain the intermediate shape.

In an embodiment, the shape memory material 114 may include metal, such as an elemental metal, an alloy, or shape memory alloy. Suitable shape memory metals may include aluminum, silver, copper, iron, nickel, zinc, tin, beryllium, or the like. Suitable shape memory alloys may include standard steels, stainless steel, carbon alloy steel, head treated steel, galvanized steel, aluminum alloys, nickel-titanium alloys (e.g., Nitinol, Ni—Ti—Cu, Ni—Ti, Co, or the like), copper-based alloys (e.g., Cu—Zn—Al, Cu—Al—Ni, Cu—Al—Sn, or the like), Co—Cr—Ni—Mo alloys (e.g., Elgiloy® or the like), or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), Polybutylmethacrylate (PBMA), poly(N, N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polypropylene, polyethylene, acrylonitrile butadiene styrene (ABS), polysulphone, or the like; Polynorbonene; other deformable polymers; or any other shape memory polymer.

The shape memory material 114 is distinct from the walls 132 of the conduit 112. The shape memory material 114 is distinct from the walls 132 of the conduit 112 when the shape memory material 114 is formed from a material that is different than the walls 132 or is otherwise distinguishable from the walls 132. When the shape memory material 114 is distinct from the walls 132 of the conduit 112, the shape memory material 114 may be disposed in selected regions of the conduit 112 rather than dispersed through the conduit 112. This allows the conduit 112 to be formed from a greater variety of materials since the materials that form the conduit 112 do not need to exhibit the properties of the shape memory material 114.

In an embodiment, the shape memory material 114 includes at least one wire (e.g., at least one rod). The wire includes a length measured along a longitudinal axis of the wire, a width measured perpendicularly to the length and a thickness measured perpendicularly to the length and the width. The length of the wire is significantly greater than the width and the thickness. In an embodiment, the shape memory material 114 includes a plate (e.g., strip). The plate exhibits a length, a width, and a thickness. The width of the plate is greater (e.g., at least 50% greater) than the thickness. The plate may initially (i.e., before shaping the plate) exhibit one or more bends therein (e.g., the plate is an at least partial tube). For example, as illustrated, the plate may exhibit a bend in the direction that is parallel to the width. The bend in the plate may be configured to correspond to the shape of the conduit 112. However, the bend in the plate may make shaping the plate more difficult. In an embodiment, the plate may be substantially planar.

As shown in FIGS. 1C and 1D, the shape memory material 114 may be at least partially disposed within the one or more walls 132 of the conduit 112. In an example, the shape memory material 114 is completely surrounded by the conduit 112 (e.g., completely disposed with the walls 132). Completely surrounding the shape memory material 114 with the conduit 112 makes it more difficult for the shape memory material 114 from becoming dislodged from the conduit 112 than if the shape memory material 114 is only partially disposed in the conduit 112. In an example, only a portion of the shape memory material 114 is surrounded by the conduit 112.

The walls 132 of the conduit 112 may exhibit a thickness measured perpendicular to a longitudinal axis of the conduit 112 (e.g., measured parallel to a radius of the conduit 112). In an embodiment, the thickness of the conduit 112 may be greater than about 0.75 mm, such as greater than about 1 mm, greater than about 1.25 mm, greater than about 1.5 mm, greater than about 1.75 mm, greater than about 2 mm, or in ranges of about 0.75 mm to about 1.25 mm, about 1 mm to about 1.5 mm, about 1.25 mm to about 1.75 mm, about 1.5 mm to about 2 mm, or about 1.75 mm to about 2.25 mm. The shape memory material 114 exhibits a thickness measure parallel to the thickness of the conduit 112. In an embodiment, the thickness of the shape memory material 114 is less than a thickness of the walls 132 of the conduit 112 which may allow the shape memory material 114 to be completely disposed in the wall 132 or, at least, improve adhesion between the walls 132 and the conduit 112. In such an embodiment, depending on the thickness of the walls 132, the shape memory material 114 may exhibit a thickness that about 0.25 mm or less, about 0.5 mm or less, about 0.75 mm or less, about 1 mm or less, about 1.5 mm or less, about 1.75 mm or less, or in ranges of about 0.25 mm to about 0.75 mm, about 0.5 mm to about 1 mm, about 0.75 mm to about 1.25 mm, about 1 mm to about 1.5, about 1.25 mm to about 1.75 mm, or about 1.5 mm to about 2 mm. It is noted that the thickness of the shape memory material 114 may be greater than about 1.75 mm, depending on the thickness of the walls 132. The thickness of the shape memory material 114 may be about 95% or less the thickness of the conduit 112, such as in ranges of about 30% to about 50%, about 40% to about 60%, about 50% to about 70%, about 60% to about 80%, or about 70% to about 90%. Generally, increasing the thickness of the shape memory material 114 may increase the ability of the shape memory material 114 to maintain a shape thereof but also increases the likelihood that the shape memory material 114 becomes dislodged from the conduit 112. As such, the thickness of the shape memory material 114 relative to the thickness of the walls 132 may be selected based on whether the ability of the shape memory material 114 to maintain its shape or prevent the shape memory material 114 becoming dislodged is preferred. In an embodiment, the thickness of the shape memory material 114 may be greater than a thickness of the walls 132, such as when the shape memory material 114 is only partially disposed in the conduit 112 and the ability of the shape memory material 114 to maintain its shape is preferred.

Disposing the shape memory material 114 in the conduit 112 may inhibit the shape memory material 114 from protruding out of the fluid impermeable barrier 102, which may cause patient discomfort and cut the skin of that patient. For example, to protrude out of the fluid impermeable barrier 102, the shape memory material 114 must become dislodged from the conduit 112, a length of the shape memory material 114 that becomes dislodged must exhibit a length that is sufficient to extend through the porous material 110, and penetrate through the fluid impermeable barrier 102. Dislodging the shape memory material 114, let alone dislodging a length of the shape memory material 114 from the conduit 112 that is sufficient to extend through the porous material 110, may be difficult, especially when the shape memory material 114 is completely disposed in the conduit 112 since it requires rupturing a portion of the conduit 112. Further, it may be difficult for the shape memory material 114 to penetrate through the fluid impermeable barrier 102. However, it is noted that the shape memory material 114 may include a terminal end that is sharp (e.g., due to cutting the shape memory material 114) which may facilitate the shape memory material 114 penetrating the fluid impermeable barrier 102. All of this makes it difficult for the shape memory material 114 to protrude from the fluid impermeable barrier 102 when the shape memory material 114 is at least partially disposed in the conduit 112, even when the fluid collection assembly 100 is mishandled.

In particular, disposing the shape memory material 114 at least partially in the conduit 112 may decrease the likelihood that the shape memory material 114 protrudes from the fluid impermeable barrier 102 than if the shape memory material 114 is disposed in other portions of the fluid collection assembly 100 (e.g., within the fluid impermeable barrier 102, between the fluid impermeable barrier 102 and the porous material 110, within the porous material 110, or between the porous material 110 and the conduit 112). For example, not disposing the shape memory material 114 in the conduit 112 eliminates the pre-requisite that the shape memory material 114 must be at least partially dislodged from the conduit 112 before the shape memory material 114 may protrude from the fluid impermeable barrier 102. Thus, not disposing the shape memory material 114 in the conduit 112 increases the likelihood that the shape memory material 114 protrudes from the fluid impermeable barrier 102.

In some embodiments, excluding the shape memory material 114, the conduit 112 may be the most rigid material in the fluid collection assembly 100 which makes the conduit 112 difficult to shape when the shape memory material 114 is spaced from the conduit 112. For example, when the shape memory material 114 is space from the conduit 112, the shape of the conduit 112 is only changed when the porous material 110 presses against the conduit 112. Pressing the porous material 110 against the conduit 112 to change the shape of the conduit 112 causes a compressive force to be applied to the porous material 110 which decreases the quantity of bodily fluids that may be stored in the porous material 110. However, at least partially disposing the shape memory material 114 in the conduit 112 causes the shape memory material 114 to control the shape of the conduit 112 instead of the porous material 110 pressing against the conduit 112. This decreases the compressive force that is applied to the porous material 110 thereby increasing the quantity of bodily fluids that may be stored in the porous material 110. The increased quantity of bodily fluids that may be stored in the porous material 110 decreases the likelihood that the porous material 110 becomes saturated with and leaks the bodily fluids.

In an embodiment, the fluid collection assembly 100 may include at least one shape memory material that is not at least partially disposed in the conduit 112 in addition to the shape memory material 114 that is at least partially disposed in the conduit 112. Examples of shape memory materials that are not disposed in the conduit 112 are disclosed in PCT International App. No. WO 2021/016026 filed on Jul. 16, 2020, the disclosure of which is incorporated herein, in its entirety, by this references.

Other embodiments of fluid impermeable barriers, fluid permeable membranes, fluid permeable supports, chambers, and their shapes and configurations are disclosed in U.S. Pat. No. 10,973,678 filed on Jun. 2, 2017; U.S. Pat. No. 10,390,989 filed on Sep. 8, 2016; and U.S. Pat. No. 10,225,376 filed on Jun. 1, 2017, the disclosure of each of which is incorporated herein, in its entirety, by this reference.

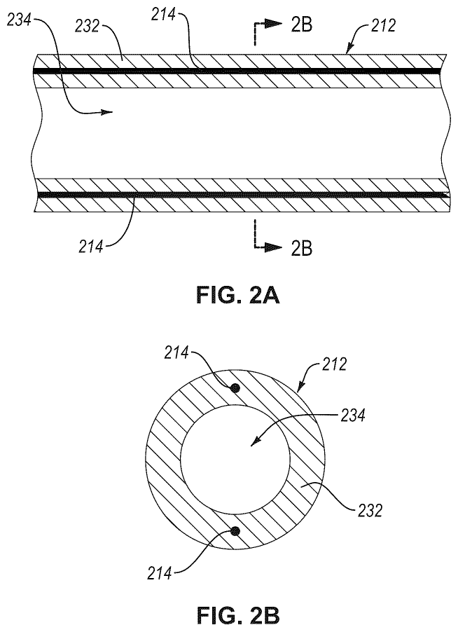

The fluid collection assemblies disclosed herein may include a plurality of shape memory materials disposed in the conduit. For example, FIG. 2A is a cross-sectional schematic of a conduit 212 that includes a plurality of shape memory materials 214 disposed therein, according to an embodiment. FIG. 2B is a cross-sectional schematic of the conduit 212 taken along plane 2B-2B shown in FIG. 2A. Except as otherwise disclosed herein, the conduit 212 and the shape memory materials 214 are the same or substantially similar to any of the conduits and shape memory materials disclosed herein, respectively. For example, the conduit 212 may include one or more walls 232 that define a passageway 234. Further, the conduit 212 and the shape memory materials 214 may be used in any of the fluid collection assemblies disclosed herein.

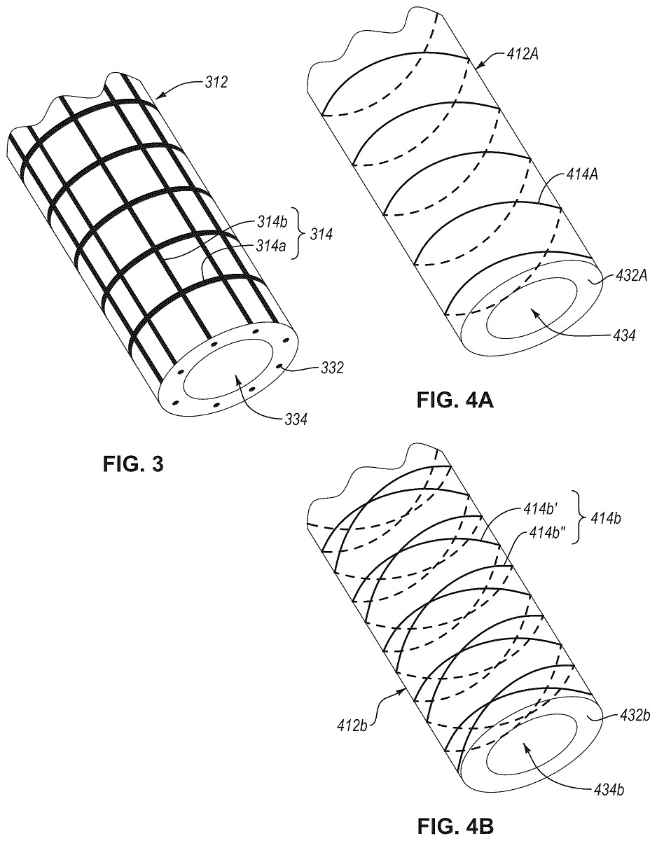

The fluid collection assembly 200 includes a plurality of shape memory materials 214 at least partially disposed in the conduit 212. For example, as illustrated, the fluid collection assembly 200 may include two shape memory materials 214 that are at least partially disposed in the conduit 212. However, it is noted that the fluid collection assembly 200 may include three or more shape memory materials 214 disposed therein, as illustrated in FIG. 3.

The plurality of shape memory materials 214, collectively, are able to maintain the shape thereof comparable to a single shape memory material 214 that exhibits a thickness that is greater than any of the plurality of shape memory materials 214. For example, the walls 232 of the conduit 212 may exhibit a thickness and each of the plurality of shape memory materials 214 may exhibit a thickness that is less than the thickness of the walls 232. However, the thickness of each of the shape memory materials 214, individually, may be unable to maintain a shape thereof during normal use of the fluid collection assembly. However, collectively, the plurality of shape memory materials 214 may be able to effectively maintain the shape thereof during normal use of the fluid collection assembly.

The shape memory materials discussed above are illustrated as extending generally parallel to a longitudinal axis of the conduit. Allowing the shape memory materials to extend generally parallel to the longitudinal axis of the conduit may facilitate formation of the conduit with the shape memory material at least partially disposed therein using a co-extrusion technique. It also minimizes a length of the shape memory material. However, shape memory materials that extend only generally parallel to the longitudinal axis may be limited in the shape that they may cause the conduit to exhibit and may be more prone to becoming dislodged from the conduit than shape memory materials that do not extend only generally parallel to the longitudinal axis. FIGS. 3-4B illustrate embodiments of shape memory materials that do not extend generally parallel to the longitudinal axis of the conduit.

FIG. 3 is an isometric view of a portion of a conduit 312 that includes at least one shape memory material 314 that does not extend generally parallel to a longitudinal axis of the conduit 312, according to an embodiment. Except as otherwise disclosed herein, the conduit 312 and the shape memory material 314 may be the same or substantially similarly to any of the conduits and shape memory materials disclosed herein, respectively. For example, the conduit 312 may include one or more walls 332 defining a passageway 334. For illustrative purposes, only the portion of the conduit 312 facing up is illustrated as being partially transparent while the portion of the conduit 312 facing down is illustrated as being opaque. It is noted that the conduit 312 may be at least one of transparent, translucent, or opaque. The conduit 312 and the shape memory material 314 may be used in any of the fluid collection assemblies disclosed herein.

The conduit 312 includes a plurality of shape memory materials 314. In an embodiment, as illustrated, the plurality of shape memory materials 314 include at least one first shape memory material 314a (e.g., a plurality of first shape memory materials 314a) and at least one second shape memory material 314b (e.g., a plurality of second shape memory materials 314b). The first shape memory material 314a extends generally parallel to the longitudinal axis of the conduit 312. The second shape memory material 314b does not extend generally parallel to the longitudinal axis of the conduit 312. For example, the second shape memory material 314b may extend perpendicular to the longitudinal axis of the conduit 312, such as extend circumferentially about the conduit 312 when the conduit 312 exhibits a generally cylindrical shape. In an embodiment, the shape memory material 314 may include at least one third shape memory material (not shown) that extends in a direction that is different than the first and second shape memory material 314a, 314b (e.g., the third shape memory material extends helically, as shown in FIGS. 4A and 4B). In an embodiment, the shape memory material 314 may only include one of the first shape memory material 314a or second shape memory material 314a.

In an embodiment, when the at least one first shape memory material 314a includes a plurality of first shape memory materials 314a, the second shape memory material 314b may intersect with the plurality of first shape memory materials 314a to form a wire mesh. In other words, the first and second shape memory materials 314a, 314b may be interconnected. In an embodiment, the second shape memory material 314b may be attached to (e.g., via welding, adhesive, etc.) to at least some of the first shape memory materials 314a that the second shape memory material 314b intersects. The interconnection between the first and second shape memory materials 314a, 314b may allow the shape memory material 314, as a whole, to exhibit more shapes and/or maintain the shape thereof better than if the shape memory material 314 only included one of the first or second shape memory material 314a, 314b. Further, the interconnection between the first and second shape memory materials 314a, 314b inhibits the first and second shape memory materials 314a, 314b from being dislodged from the conduit 312 since dislodging one would require dislodging the other or breaking the interconnection therebetween.

The conduit 312 may be formed using any suitable technique. In an embodiment, the first and second shape memory materials 314a, 314b may be attached to each other to maintain the positioned thereof relative to each other. After attaching the first and second shape memory materials 314a, 314b together, the first and second shape memory materials 314a, 314b may be co-extruded with the material that forms the conduit 312. It is noted that the second shape memory materials 314b may not extend completely around a circumference of the conduit 312 to allow extruder to support a portion of the die that forms the passageway 334. In an embodiment, the first and second shape memory materials 314a, 314b may be positioned relative to each other according to their desired final configuration. The first and second shape memory materials 314a, 314b may then be disposed in a mold and the material that forms the conduit 312 may be injected into the mold.

The shape memory materials may extend within the conduit in one or more directions that are not parallel and/or perpendicular to a longitudinal axis of the conduit. For example, FIG. 4A is an isometric view of a portion of a conduit 412a that includes at least one shape memory material 414a, according to an embodiment. Except as otherwise disclosed herein, the conduit 412a and the shape memory material 414a may be the same or substantially similarly to any of the conduits and shape memory materials disclosed herein, respectively. For example, the conduit 412a may include one or more walls 432a defining a passageway 434a. The conduit 412a is illustrated as being transparent for illustrative purposes, though it is noted that the conduit 412a may be opaque. Also, for illustrative purposes, the portions of the shape memory material 414a disposed in the portions of the conduit 412a facing down are illustrated using dashed lines. The conduit 412a and the shape memory material 414a may be used in any of the fluid collection assemblies disclosed herein.

The shape memory material 414a extends generally helically relative to the longitudinal axis of the conduit 412a. Such a shape memory material 414a may be more difficult to be dislodged from the conduit 412a than if the shape memory material 414a extended generally parallel to the longitudinal axis of the conduit 412a. Further, the shape memory material 414a may be formed into more shapes, such as more complex shapes, then if the shape memory material 414a extended generally parallel to the longitudinal axis of the conduit 412a.

The shape memory material 414a may be disposed in the conduit 412a using any suitable technique. In an embodiment, the shape memory material 414a may be disposed in the conduit 412a using a co-extruding technique. In such an embodiment, the shape memory material 414a may be feed through a die of the extruder in a direction that is non-parallel to the flow of the material that forms the conduit 412a. Feeding the shape memory material 414a into the die in such an non-parallel direction may cause the shape memory material 414a to exhibit the helical shape. In an embodiment, the shape memory material 414a may be forced into a helical shape, positioned in a die, and the material that forms the conduit 412a may be injected into the mold.

FIG. 4B is an isometric view of a portion of a conduit 412b that includes a plurality of shape memory materials 414b at least partially disposed therein, according to an embodiment. Except as otherwise disclosed herein, the conduit 412b and the shape memory materials 414b may be the same or substantially similarly to any of the conduits and shape memory materials disclosed herein, respectively. For example, the conduit 412b may include one or more walls 432b defining a passageway 434b. The conduit 412b is illustrated as being transparent for illustrative purposes, though it is noted that the conduit 412b may be opaque. Also, for illustrative purposes, the portions of the shape memory material 414b disposed in the portions of the conduit 412b facing down are illustrated using dashed lines. The conduit 412b and the shape memory materials 414b may be used in any of the fluid collection assemblies disclosed herein.

The plurality of shape memory materials 414b includes at least a first shape memory material 414b′ and a second shape memory material 414b″. At least one of the first or second shape memory material 414b′, 414b″ is at least partially disposed in the conduit 412b. The first shape memory material 414b′ and the second shape memory material 414b″ extend generally helically along the longitudinal axis of the conduit 412b. In an embodiment, as shown, the first shape memory 414b′ and the second shape memory material 414b″ exhibit different chiralities. For example, one of the first shape memory material 414b′ or the second shape memory material 414b″ forms a left-handed helix and the other of the first shape memory material 414b′ or the second shape memory material 414b″ forms a right-handed helix. The different chiralities of the first and second shape memory materials 414b′, 414b″ cause the first and second shape memory materials 414b′, 414b″ to intersect which may inhibit dislodgement of the first and second shape memory materials 414b′, 414b″ from the conduit 412b. The first and second shape memory materials 414b′, 414b″ having different chiralities may be formed via a co-extrusion process, for example, by feeding two different sources into the die of the extruder at different angles relative to the flow path of the material that forms the conduit 412b. It is noted that the first and second shape memory materials 414b′, 414b″ having different chiralities may be formed via other techniques, such as an injection molding technique. In an embodiment, the first shape memory 414b′ and the second shape memory material 414b″ exhibit the same chirality.

It is noted that, when the shape memory material includes a plurality of shape memory materials, the plurality of shape memory materials may include any combination of shape memory materials disclosed herein. For example, the plurality of shape memory materials may include at least two of at least one shape memory material that extends generally parallel to the longitudinal axis of the conduit, at least one shape memory material that extends circumferentially, or at least one shape memory material that extends helically.

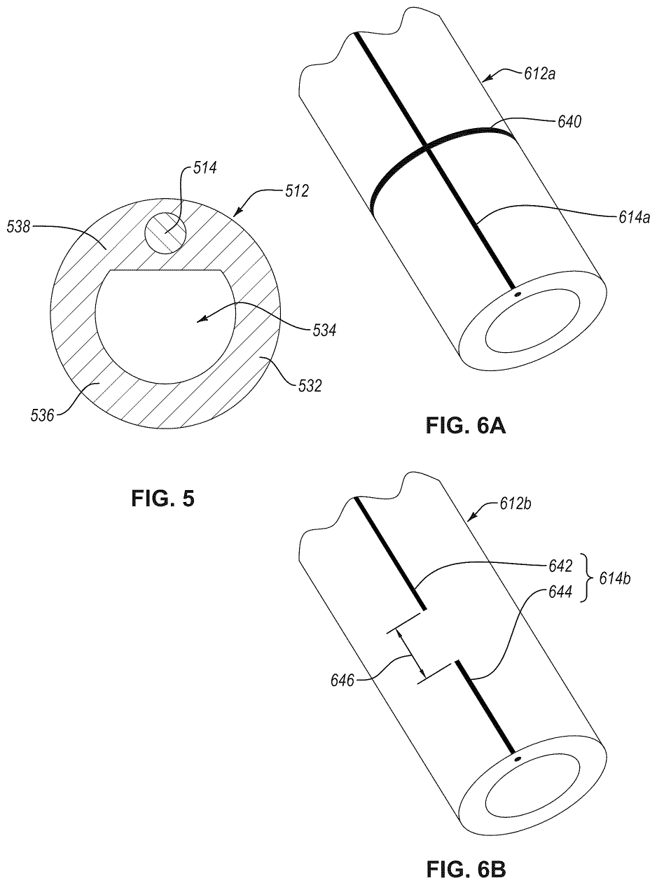

FIG. 5 is a cross-sectional schematic of a portion of a conduit 512 that includes at least one shape memory material 514 disposed therein, according to an embodiment. Except as otherwise disclosed herein, the conduit 512 and the shape memory material 514 is the same or substantially similar to any of the conduits and shape memory materials, respectively, disclosed herein. The conduit 512 and the shape memory material 514 may be used in any of the fluid collection assemblies disclosed herein.

The conduit 512 includes one or more walls 532 and the thickness of the walls 532 may vary. For example, the one or more walls 532 may include a first portion 536 and a second portion 538. The second portion 538 may exhibit a thickness that is greater than the first portion 536. The greater thickness of the second portion 538 allows the second portion 538 to receive a shape memory material 514, for example, exhibiting at least one of a thickness that is greater than the thickness of the first portion 536 (e.g., allowing the shape memory material 514 to be completely disposed in the second portion 538) or a thickness that is sufficient great that it is difficult to maintain the shape memory material 514 attached to the first portion 536 during normal use of the fluid collection assembly.

For example, the first portion 536 of the walls 532 may exhibit a thickness that is similar to the walls of conduits that are commonly used in fluid collection assemblies (e.g., a thickness of about 1 mm to about 2 mm). The shape memory material 514 may exhibit a thickness that is sufficiently great that at least partially disposing the shape memory material 514 in the first portion 536 is not practical. One solution is to increase the thickness of the entirety of the conduit 512. However, increasing the thickness of the entirety of the conduit 512 may at least one of make the conduit 512 sufficient rigid that the shape memory material 514 struggles to change a shape thereof, decreases the volume of the chamber that may store the bodily fluids, or decrease the diameter of the passageway 534 which may decrease the rate at which the bodily fluids may be removed from the chamber. However, increasing the thickness of only a portion of the conduit 512 allows the shape memory material 514 to be securely attached to the conduit 512 (e.g., completely disposed within the walls 532) while minimizing the adverse effects of increasing the thickness of the entirety of the walls 532.

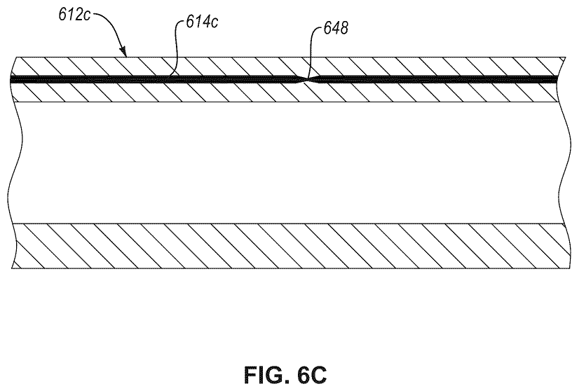

In some embodiments, a fluid collection assembly may include a conduit, with the at least one shape memory material at least partially disposed therein, exhibiting a length that is about 12.5 cm or greater, about 15 cm or greater, about 17.5 cm or greater, about 20 cm or greater, about 22.5 cm or greater, about 25 cm or greater, about 30 cm or greater, about 40 cm or greater, about 50 cm or greater, about 75 cm or greater, about 1 m or greater, about 2 m or greater, or in ranges of about 12.5 cm to about 9.5 cm, about 15 cm to about 20 cm, about 17.5 cm to about 22.5 cm, about 20 cm to about 25 cm, about 22.5 cm to about 30 cm, about 25 cm to about 40 cm, about 30 cm to about 50 cm, about 40 cm to about 75 cm, 50 cm to about 1 m, or about 75 cm to about 2 m. It is noted that the conduit may extend from the fluid impermeable barrier and, as such, the conduit may exhibit a length that is greater than the fluid impermeable barrier. However, the conduit with the at least one shape memory material at least partially disposed therein (as formed and/or provided) may exhibit a length that is greater than the length of the conduit that is used in the fluid collection assembly. For example, the conduit with the at least one shape memory material at least partially disposed therein may be formed or provided while exhibiting a length that is about 1 m or greater, about 5 m or greater, about 10 m or greater, about 50 m or greater, or about 100 m or greater. As such, the as-formed and/or as-provided conduit may need to be cut into a plurality of conduits, wherein at least one of the plurality of conduits formed from cutting the as-formed and/or as-provided conduit is used in a fluid collection assembly. FIGS. 6A-6C illustrate different features of the as-formed and/or as-provided conduit that may facilitate cutting the as-formed and/or as-provided conduit.

FIG. 6A is an isometric view of a portion of a conduit 612a that includes at least one shape memory material 614a at least partially disposed therein, according to an embodiment. The conduit 612a is illustrated as being partially transparent for illustrative purposes, but may be at least partially opaque. Except as otherwise disclosed herein, the conduit 612a and the shape memory material 614a may be the same or substantially similar to any of the conduits and shape memory materials disclosed herein. A portion of the illustrated conduit 612a and the shape memory material 614a may be used in any of the fluid collection assemblies disclosed herein after cutting the conduit 612a and the shape memory material 614a.

The conduit 612a, as-formed and/or as-provided, exhibits a length that is greater than what is required to be disposed in a fluid collection assembly (not shown). As such, the conduit 612a may need to be cut. To facilitate cutting of the conduit 612a, the conduit 612a may include at least one marking 640 formed thereon. The marking 640 indicates a location on the conduit 612a that is to be cut. In an example, the marking 640 may indicate a location on the conduit 612a to be cut such that separated portion of the conduit 612a exhibits a desired length to be used with a fluid collection assembly. In an example, the marking 640 may indicate a location of the conduit 612a having one or more feature that facilitate cutting of the conduit 612a, such as a gap between adjacent shape memory materials 614a (as shown in FIG. 6B) or a crimped portion of the shape memory material 614 (as shown in FIG. 6C).

In an example, the marking 640 may be formed by painting or printing on a portion of the conduit 612a. In an example, the marking 640 may be a recess (e.g., divot) or protrusion that is formed on the conduit 612a. Regardless of the method used for form the marking 640, the marking 640 may be formed at some point after forming the conduit 612a, such as soon after extruding the conduit 612a from a die. It is noted that at least a portion of the marking 640 may remain on the conduit 612a after cutting the conduit 612a. For example, the conduit 612a may include at least one paint or printing thereon, may include a thinned region when the marking 640 is a recess, or a thickened region when the marking 640 is a protrusion.

As previously discussed, the shape memory materials at least partially disposed in the conduit may be formed from a metal or other hard materials. Such a shape memory material may make cutting the conduit with the shape memory material disposed therein difficult. For example, cutting the shape memory material may require the use of advanced cutting tools and/or may require more regularly replacement of the cutting tool (e.g., due to dulling of the cutting tool). As such, the shape memory materials disclosed herein may include one or more features that allows facilitates cutting the shape memory material. FIGS. 6B and 6C illustrate examples of features of the shape memory materials that may facilitate cutting thereof.

FIG. 6B is an isometric view of a portion of a conduit 612b that includes at least one shape memory material 614b at least partially disposed therein, according to an embodiment. The conduit 612b is illustrated as being transparent for illustrative purposes but may be at least partially opaque. Except as otherwise disclosed herein, the conduit 612b and the shape memory material 614b may be the same or substantially similar to any of the conduits and shape memory materials disclosed herein, respectively. The conduit 612b and the shape memory material 614b, after cutting, may be used in any of the fluid collection assemblies disclosed herein.

The shape memory material 614b is formed from a plurality of segments. For example, as illustrated, the shape memory material 614b may be formed from at least a first segment 642 and a second segment 644. The first segment 642 and the second segment 644 are distinct from each other. The first segment 642 may be spaced from the second segment 644 along the longitudinal axis of the conduit 612b by a gap 646. The gap 646 may provide a location to cut the conduit 612b without having to also cut the shape memory material 614b since the shape memory material 614b may be difficult to cut and/or may damage (e.g., dull) the tool used to cut the conduit 612b. The first segment 642 may exhibit a length that is the same or slightly smaller than the desired length of the conduit 612b that is disposed in a fluid collection assembly. The location of the gap 646 may be indicated by a marking (not shown), as previously discussed.

In an embodiment, the shape memory material 614b is disposed in the conduit 612b using a co-extrusion technique. For example, the first and second segments 642, 644 may be attached together with a material (e.g., the same material that forms the conduit 612b) that is easier to cut than the first and second segments 642, 644. Connecting the first and second segments 642, 644 together with the material allows the first and second segments 642, 644 to be spooled into the die. It is noted that the first and second segments 642, 644 do not need to be connected together to be co-extruded. In an embodiment, the shape memory material 614b is at least partially disposed in the conduit 612b by positioning the shape memory material 614b (e.g., the first and second segments 642, 644) in a mold and the material that forms the conduit 612b is injected into the mold.

FIG. 6C is an cross-sectional schematic of a portion of a conduit 612c that includes at least one shape memory material 614c at least partially disposed therein, according to an embodiment. The conduit 612c is illustrated as being transparent for illustrative purposes but may be at least partially opaque. Except as otherwise disclosed herein, the conduit 612c and the shape memory material 614c may be the same or substantially similar to any of the conduits and shape memory materials disclosed herein, respectively. The conduit 612c and the shape memory material 614c, after cutting, may be used in any of the fluid collection assemblies disclosed herein.

The shape memory material 614c exhibits a thickness measured traverse to a longitudinal axis of the conduit 612c (e.g. measured radially). The thickness of the shape memory material 614c may be selectively varied. The variation of the thickness of the shape memory material 614c may vary to facilitate cutting of the shape memory material 614c. For example, the shape memory material 614c may include at least one narrowed region 648 that exhibits a thickness that is less than the portions of the shape memory material 614c thereabout. The narrowed region 648 may facilitate cutting the shape memory material 614c since the cutting tool only needs to cut through a smaller thickness of the shape memory material 614c. For instance, the narrowed region 648 may exhibit a smaller cross-sectional area than the portions of the shape memory material 614c thereabout or may engage a greater surface area of the cutting tool while being cut thereby better distributing wear across the cutting tool.

The narrowed region 648 may be formed by crimping the shape memory material 614c, grinding, otherwise mechanically removing portions of the shape memory material 614c, or otherwise thinning at least one portion of the shape memory material 614c. The narrowed region 648 may be formed before at least partially disposing the shape memory material 614c in the conduit 612c.

Changing the shape of the shape memory materials disclosed herein, for example, from the first shape to the second shape, causes the conduit to change a shape thereof. In some embodiments, changing the shape of the conduit presses against the porous material to change the shape of the porous material. Further, changing the shape of the porous material also causes the porous material to press against the fluid impermeable barrier to change the shape of the fluid impermeable barrier. Generally, the porous material and the fluid impermeable barrier resist a change in the shape thereof less than the conduit. However, the porous material and the fluid impermeable barrier do resist changes in the shape thereof which causes a compressive force to be applied to the porous material. The compressive force applied to the porous material may decrease the quantity of bodily fluids that may be stored in the porous material which increases the likelihood that the porous material becomes saturated with bodily fluids and leaks.

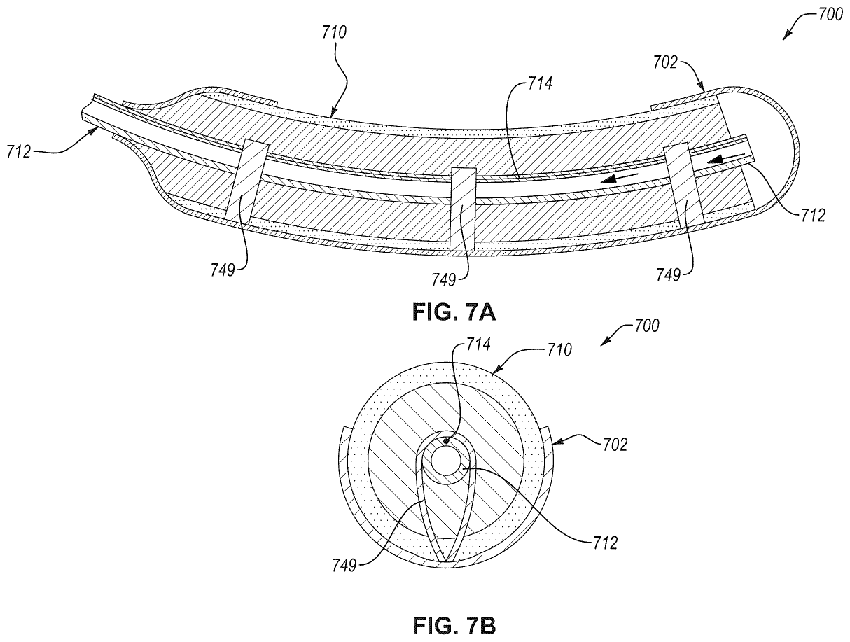

The fluid collection assemblies disclosed herein may include one or more structure that are configured to limit the compressive force applied to the porous material. In particular, the structures may be configured to transfer the change of shape of the conduit to the fluid impermeable barrier substantially without using the porous material as an intermediary. FIG. 7A is a cross-sectional schematic of a fluid collection assembly 700, according to an embodiment. FIG. 7B is a cross-sectional schematic of the fluid collection assembly 700 taken along plane 7B-7B of FIG. 7A. Except as otherwise disclosed herein, the fluid collection assembly 700 is the same or substantially similar to any of the fluid collection assemblies disclosed herein. For example, the fluid collection assembly 700 includes a fluid impermeable barrier 702, a porous material 710, a conduit 712, and a shape memory material 714 at least partially disposed in the conduit 712.

The fluid collection assembly 700 includes at least one brace 749 that is configured to force the fluid impermeable barrier 702 to exhibit a shape change that corresponds more closely to the shape change of the shape memory material 714 (e.g., the shape change of the conduit 712) than if the fluid collection assembly 700 did not include the brace 749. The brace 749 also minimizes the compressive force applied to the porous material 710. The brace 749 is connected to (e.g., extends around) the conduit 712 The brace 749 extends from the conduit 712 to the fluid impermeable barrier 702. The brace 749 is attached to the fluid impermeable barrier 702, such as with an adhesive. To allow the brace 749 to extend from the conduit 712 to the fluid impermeable barrier 702, one or more components of the fluid collection assembly 700 (e.g., the porous material 710) may include one or more slits formed therein through which the brace 749 extend. The brace 749 may be configured to transfer a shape change in the shape memory material 714 to the fluid impermeable barrier 702 such that the fluid impermeable barrier 702 exhibits a shape change that substantially corresponds to the shape change of the shape memory material 714.

The fluid collection assembly 700 may include any suitable number of braces 749, such as 1, 2, 3, 4, 5, 6, 7, 8, 7, 10, or greater than 10 braces 749. Generally increasing the number of braces 749 causes the fluid impermeable barrier 702 to more accurately correspond to the shape of the shape memory material 714 and decreases the normal force that is applied to the porous material 710.

It is noted that the braces 749 may not always cause the fluid impermeable barrier 702 to exhibit the same shape change as the shape memory material 714. For example, when the braces 749 are looped around and are not attached to the conduit 712 (as shown), the braces 749 may only cause the fluid impermeable barrier 702 to exhibit the shape change of the shape memory material 714 when the braces 749 pull (e.g., the braces 749 are in tension) the fluid impermeable barrier 702 towards the conduit 712 but not when the braces 749 push (e.g., the braces 749 are in compression) the fluid impermeable barrier 702 away from the conduit 712.

In an embodiment, the braces 749 are formed from any of the shape memory materials disclosed herein. In an embodiment, the braces 749 may be formed from a non-shape memory material, such as fabric.

FIG. 8 is a block diagram of a system 850 for fluid collection, according to an embodiment. The system 850 includes a fluid collection assembly 800, a fluid storage container 852, and a vacuum source 854. The fluid collection assembly 800, the fluid storage container 852, and the vacuum source 854 may be fluidly coupled to each other via one or more conduits 856. At least a portion of the conduit 856 may include at least one shape memory material at least partially disposed therein, such as a portion of the conduit 856 that is disposed in and forms part of. the fluid collection assembly 800. In some embodiments, a portion of the conduit 856 (e.g., the portions of the conduit 856 spaced from the fluid collection assembly 800) do not include at least one shape memory material disposed therein.

The fluid collection assembly 800 may be operably coupled to one or more of the fluid storage container 852 or the vacuum source 854 via the conduit 856. Fluid (e.g., urine or other bodily fluids) collected in the fluid collection assembly 800 may be removed from the fluid collection assembly 800 via the conduit 856 which protrudes into the fluid collection assembly 800. For example, an inlet of the conduit 856 may extend into the fluid collection assembly 800, such as to a fluid reservoir thereof. The outlet of the conduit 856 may extend into the fluid collection assembly 800 or the vacuum source 854. Suction force may be introduced into the chamber of the fluid collection assembly 800 via the inlet of the conduit 856 responsive to suction (e.g., vacuum) force applied at the outlet of the conduit 856.

The suction force may be applied to the outlet of the conduit 856 by the vacuum source 854 either directly or indirectly. The suction force may be applied indirectly via the fluid storage container 852. For example, the outlet of the conduit 856 may be disposed within the fluid storage container 852 and an additional conduit 856 may extend from the fluid storage container 852 to the vacuum source 854. Accordingly, the vacuum source 854 may apply suction to the fluid collection assembly 800 via the fluid storage container 852. The suction force may be applied directly via the vacuum source 854. For example, the outlet of the conduit 856 may be disposed within the vacuum source 854. An additional conduit 856 may extend from the vacuum source 854 to a point outside of the fluid collection assembly 800, such as to the fluid storage container 852. In such examples, the vacuum source 854 may be disposed between the fluid collection assembly 800 and the fluid storage container 852.

The fluid collection assembly 800 may be similar or identical to any of the fluid collection assemblies disclosed herein in one or more aspects. The fluid collection assembly 800 may be shaped and sized to be positioned adjacent to a female urethral opening. For example, the fluid collection assembly 800 may include a fluid impermeable barrier at least partially defining a chamber (e.g., interior region) of the fluid collection assembly 800. The fluid impermeable barrier also defines an opening extending therethrough from the external environment. The opening may be positioned adjacent to a female urethral opening. The fluid collection assembly 800 may include may include at least one porous material disposed in the chamber such as one or more of a fluid permeable support and a fluid permeable membrane. The fluid collection assembly 800 includes the shape memory material at least partially disposed in a conduit (e.g., conduit 856) that is at least partially disposed in the chamber. The shape memory material is sized, shaped, and positioned to retain a selected geometric configuration as disclosed herein. The conduit 856 may extend into the fluid collection assembly 800, through one or more of the fluid impermeable barrier, fluid permeable membrane, or the fluid permeable support to a distal end region of the fluid collection assembly 800. The conduit 856 includes an inlet and an outlet, the outlet being fluidly coupled to the fluid storage container 852 and the inlet being positioned in a portion of the chamber selected to be at a gravimetrically low point of the fluid collection assembly 800 when worn.

The fluid storage container 852 is sized and shaped to retain a fluid therein. The fluid storage container 852 may include a bag (e.g., drainage bag), a bottle or cup (e.g., collection jar), or any other enclosed container for storing the bodily fluids, such as urine. In some examples, the conduit 856 may extend from the fluid collection assembly 800 and attach to the fluid storage container 852 at a first point therein. An additional conduit 856 may attach to the fluid storage container 852 at a second point thereon and may extend and attach to the vacuum source 854. Accordingly, a vacuum (e.g., suction) may be drawn through fluid collection assembly 800 via the fluid storage container 852. The bodily fluids may be drained from the fluid collection assembly 800 using the vacuum source 854.

The vacuum source 854 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 854 may provide a vacuum or suction to remove fluid from the fluid collection assembly 800. In some examples, the vacuum source 854 may be powered by one or more of a power cord (e.g., connected to a power socket), one or more batteries, or even manual power (e.g., a hand operated vacuum pump). In some examples, the vacuum source 854 may be sized and shaped to fit outside of, on, or within the fluid collection assembly 800. For example, the vacuum source 854 may include one or more miniaturized pumps or one or more micro pumps. The vacuum sources 854 disclosed herein may include one or more of a switch, a button, a plug, a remote, or any other assembly suitable to activate the vacuum source 854.

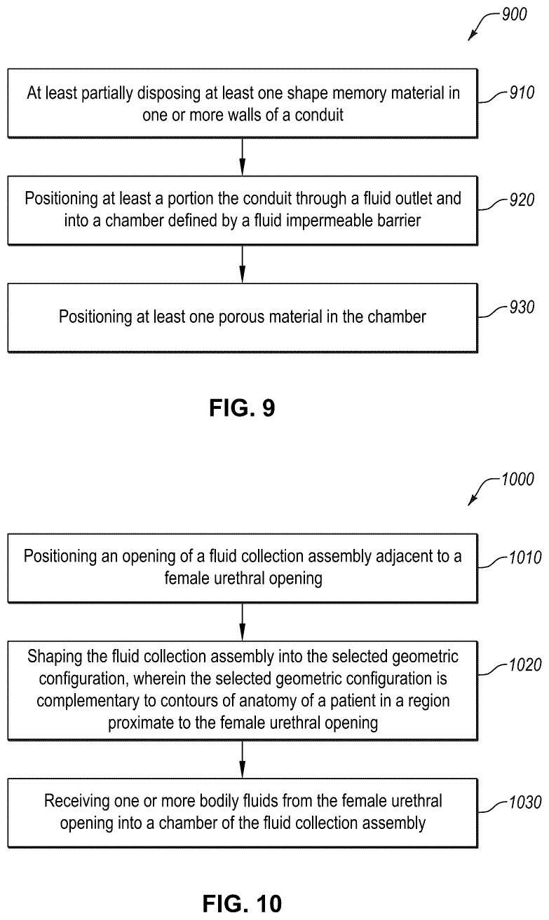

FIG. 9 is flow diagram of a method 900 to form any of the fluid collection assemblies disclosed herein. The method 900 may include act 910, which recites “at least partially disposing at least one shape memory material in one or more walls of a conduit.” Act 910 may be followed by act 920, which recites “positioning at least a portion the conduit through a fluid outlet and into a chamber defined by a fluid impermeable barrier.” Act 920 may be followed by act 930, which recites “positioning at least one porous material in the chamber.”

Acts 910, 920, 930 of the method 900 are for illustrative purposes. For example, the act 910, 920, 930 of the method 900 may be performed in different orders, split into multiple acts, modified, supplemented, or combined. In an example, one or more of the acts 910, 920, 930 of the method 900 may be omitted from the method 900.

Act 910 recites “at least partially disposing at least one shape memory material in one or more walls of a conduit.” The shape memory material may be disposed in the one or more walls of the conduit using any of the techniques disclosed herein or any other suitable technique. In an example, the shape memory material may be disposed in the one or more walls of the conduit using a co-extrusion technique that includes co-extruding at least one polymer that forms the conduit and the at least one shape memory material. The co-extrusion technique may include feeding the shape memory material into a die in a direction that is substantially parallel to the flow of the polymer if the shape memory material extends parallel to the longitudinal axis of the conduit. The co-extrusion technique may include feeding the shape memory material into a die in a direction that is not parallel to the flow of the polymer if the shape memory material does not extend parallel to the longitudinal axis of the conduit (e.g., the shape memory material is helical). In an example, the shape memory material may be disposed in the conduit using an injection molding technique. The injection molding technique may include positioning the shape memory material in a mold and injecting the one or more polymers that form the conduit into the mold.

In an embodiment, act 910 includes forming a conduit that needs to be cut because the conduit, as formed and/or as provided, exhibits a length that is too long for a single fluid collection assembly. In such an embodiment, the method 900 may include cutting the conduit into two or more pieces wherein only one of the pieces is configured to be positioned through the fluid outlet and into the chamber of each fluid collection assembly. Cutting the conduit may include cutting one or more markings formed on the conduit, cutting the conduit at a gap between two segments of the shape memory material, or cutting the shape memory material at a narrowed region thereof.

After act 910, the method 900 includes acts 920 and 930. Act 920 recites “positioning at least a portion the conduit through a fluid outlet and into a chamber defined by a fluid impermeable barrier” and act 930 recites “positioning at least one porous material in the chamber.” Acts 920 and 930 may be performed in any order or may be performed simultaneously.

FIG. 10 is a flow diagram of a method 1000 to collect one or more bodily fluids, according to an embodiment. The method 1000 of collecting bodily fluids may utilize use any of the fluid collection assemblies and/or fluid collection systems disclosed herein. The method 1000 may include act 1010, which recites “positioning an opening of a fluid collection assembly adjacent to a female urethral opening.” Act 1010 may be followed by act 1020, which recites “shaping the fluid collection assembly into the selected geometric configuration, wherein the selected geometric configuration is complementary to contours of anatomy of a patient in a region proximate to the female urethral opening.” Act 1020 may be followed by act 1030, which recites “receiving one or more bodily fluids from the female urethral opening into a chamber of the fluid collection assembly.”

Acts 1010, 1020, 1030 of the method 1000 are for illustrative purposes. For example, the act 1010, 1020, 1030 of the method 1000 may be performed in different orders, split into multiple acts, modified, supplemented, or combined. In an example, one or more of the acts 1010, 1020, 1030 of the method 1000 may be omitted from the method 1000. Any of the acts 1010, 1020, or 1030 may include using any of the fluid collection assemblies or systems disclosed herein.

Act 1010 recites “positioning an opening of a fluid collection assembly adjacent to a female urethral opening.” The act 1010 of positioning an opening of a fluid collection assembly adjacent to a female urethral opening may include utilizing any of the fluid collection assemblies or systems disclosed herein. In some examples, act 1010 may include positioning the opening of a female fluid collection assembly such that the fluid permeable membrane of the female fluid collection assembly abuts or is positioned proximate to the female urethral opening. In some examples, positioning an opening of a fluid collection assembly adjacent to a female urethral opening may include positioning the opening over the female urethral opening, such as positioning a longitudinally extending opening of the fluid collection assembly over the female urethral opening.

Act 1020 recites “shaping the fluid collection assembly into the selected geometric configuration, wherein the selected geometric configuration is complementary to contours of anatomy of a patient in a region proximate to the female urethral opening.” Shaping the fluid collection assembly into the selected geometric configuration may include forming the fluid collection assembly into a second shape that is different than a first shape or vice versa. Shaping the fluid collection assembly into the selected shape may include shaping a female fluid collection assembly to contour to the anatomy around the urethral opening of a female patient. In some embodiments, shaping the fluid collection assembly into the selected geometric configuration includes forming the (e.g., a longitudinal shape of the) fluid collection assembly into an arcuate shape conforming to the perineal region of the patient. For example, shaping the fluid collection assembly into the selected geometric configuration may include forming the fluid collection assembly into an arcuate shape conforming to the vaginal and perineal region of a patient. In some embodiments, shaping the fluid collection assembly into the selected geometric configuration includes flattening or rounding a lateral cross-section of the fluid collection assembly.

Shaping the fluid collection assembly into the selected geometric configuration may include manually bending, stretching, compressing, or otherwise manipulating at least one portion of the fluid collection assembly to the selected geometric configuration. In some embodiments, shaping the fluid collection assembly into the selected geometric configuration includes flattening or rounding a lateral cross-section of the fluid collection assembly. In some embodiments, shaping the fluid collection assembly into the selected geometric configuration includes compressing or bending a the fluid collection assembly longitudinally.

Act 1030 recites, “receiving one or more bodily fluids from the female urethral opening into a chamber of the fluid collection assembly.” In some examples, receiving fluid from the female urethral opening into a chamber of the fluid collection assembly includes receiving the fluid through the opening of the fluid collection assembly. Receiving fluid from the female urethral opening into a chamber of the fluid collection assembly may include wicking the bodily fluids away from the opening using porous material, such as via a fluid permeable membrane and a fluid permeable support. Receiving fluid from the female urethral opening into a chamber of the fluid collection assembly may include flowing the bodily fluids towards a portion of the chamber that is fluidly coupled to an inlet of a conduit in fluid communication a vacuum source. For instance, receiving the bodily fluid from the female urethral opening into a chamber of the fluid collection assembly may include flowing the bodily fluids to a substantially unoccupied portion of the chamber (e.g., a reservoir), to a gravimetrically low point of the chamber, etc., such as via gravity, wicking, or suction force. In some examples, wicking the fluid into the chamber via the fluid permeable membrane and fluid permeable support may include wicking urine into a reservoir in the fluid collection assembly.

The method 1000 may include applying suction with a vacuum source effective to suction the bodily fluids from the chamber via a conduit disposed therein and fluidly coupled to the vacuum source may include using any of the vacuum sources disclosed herein. Applying suction with a vacuum source may include activating the vacuum source (e.g., suction assembly) in fluid communication with the inlet of the conduit in the fluid collection assembly. In some examples, activating the vacuum source in fluid communication with the inlet of the conduit in the fluid collection assembly may include supplying power to the vacuum source by one or more of flipping an on/off switch, pressing a button, plugging the vacuum source into a power outlet, putting batteries into the vacuum source, etc. In some examples, the vacuum source may include a hand operated vacuum pump and applying suction with a vacuum source may include manually operating the hand operated vacuum pump effective to suction the bodily fluids from the chamber via the conduit disposed therein that is fluidly coupled to the vacuum source.

In some examples, applying suction with a vacuum source effective to suction the bodily fluids from the chamber via a conduit disposed therein and fluidly coupled to the vacuum source may be effective to remove at least some bodily fluids (e.g., urine) from the chamber (e.g., interior region) of the fluid collection assembly. In some examples, applying suction with a vacuum source effective to suction the bodily fluids from the chamber via a conduit disposed therein and fluidly coupled to the vacuum source may be effective to transfer at least some of the bodily fluids from the chamber to a fluid storage container (e.g., a bottle or bag), such as from one or more of a reservoir, fluid permeable support, or fluid permeable membrane.

In some examples, the vacuum source (e.g., suction assembly) may be disposed on or within the fluid collection assembly and applying suction with the vacuum source may include activating the vacuum source. In some examples, the vacuum source may be spaced from the fluid collection assembly and applying suction with the vacuum source may include activating the vacuum source.

In some examples, applying suction with a vacuum source effective to suction the bodily fluids from the chamber via a conduit disposed therein and fluidly coupled to the vacuum source may include detecting moisture in the chamber (e.g., via one or more moisture sensors) and responsive thereto, activating the vacuum source to provide suction in the chamber. The control of the vacuum source responsive to the signals indicating that moisture or a level thereof is present in the chamber may be automatic, such as via a controller (e.g., computer programmed to perform the operation), or may merely provide an indication that a level of moisture is present that may necessitate removal of the bodily fluids from the chamber of the fluid collection assembly. In the latter case, a patient may receive the indication (e.g., from the controller) and activate the vacuum pump manually.

In an example, the method 1000 may include collecting the bodily fluids that are removed from the fluid collection assembly, such as into a fluid storage container that is spaced from the fluid collection assembly and fluidly coupled to the conduit. The fluid storage container may include any of the fluid storage containers disclosed herein.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. (canceled)

2. A fluid collection assembly, comprising:
a fluid impermeable barrier including a chamber at least partially therein, at least one opening configured to be positioned adjacent to a female urethral opening, and a fluid outlet;
at least one porous material disposed in the chamber;
a conduit extending from the fluid outlet into the chamber, the conduit including one or more walls that define a passageway; and
at least one shape memory material completely disposed within the one or more walls of the conduit, the at least one shape memory material being sized, shaped, and positioned in the one or more walls to retain a selected geometric configuration, the at least one shape memory material includes a first shape memory material, the first shape memory material disposed in a portion of the conduit that is opposite the at least one opening.

3. The fluid collection assembly of claim 2, wherein the conduit includes an inner surface defining the passageway and an outer surface opposite the inner surface, wherein an entirety of the outer surface of the conduit extending into the chamber is circular in a cross-sectional plane that is perpendicular to a longitudinal axis of the conduit.

4. The fluid collection assembly of claim 2, wherein the conduit includes one or more markings thereon configured to facilitate at least one of correct insertion depth or correct rotational orientation of the conduit relative to the chamber.

5. The fluid collection assembly of claim 2, wherein the one or more walls of the conduit includes a first portion and a second portion, the first portion configured to receive the first shape memory material, the first portion exhibiting a thickness that is different than a thickness of the second portion, the thickness of the first portion and the thickness of the second portion measured from the outer surface of the one or more walls to the immediately adjacent inner surface, the thickness of the first portion and the thickness of the second portion remains substantially constant along the longitudinal axis of the conduit.

6. The fluid collection assembly of claim 5, wherein the first shape memory material exhibits a thickness that is greater than a thickness of the second portion.

7. The fluid collection assembly of claim 2, wherein the first shape memory material exhibits a thickness of about 1.5 mm or less.

8. The fluid collection assembly of claim 2, wherein the first shape memory material exhibits a thickness that is less than a thickness of the conduit.

9. The fluid collection assembly of claim 2, wherein the at least one shape memory material includes a single wire.

10. The fluid collection assembly of claim 2, wherein the at least one shape memory material includes a plurality of wires.

11. The fluid collection assembly of claim 2, wherein the at least one shape memory material includes a plate exhibiting a length, a width, and a thickness, the width of the plate being at least 50% greater than the thickness.

12. The fluid collection assembly of claim 2, wherein the at least one shape memory material further includes a second shape memory material.

13. The fluid collection assembly of claim 12, wherein the first shape memory material and the second shape memory material intersect.

14. The fluid collection assembly of claim 12, wherein the first shape memory material includes a metal and the second shape memory material includes a polymer, wherein the second shape memory material is distinct and separate from the conduit and the polymer is deformable.

15. The fluid collection assembly of claim 12, wherein the second shape memory material extends generally helically.

16. The fluid collection assembly of claim 2, wherein the first shape memory material extends substantially parallel to a longitudinal axis of the conduit.

17. The fluid collection assembly of claim 2, wherein the first shape memory material includes two terminal ends longitudinally spaced from each other, each of the two terminal ends spaced from adjacent terminal ends of the conduit.

18. The fluid collection assembly of claim 2, wherein the fluid impermeable barrier and the at least one porous material define a fluid reservoir that is substantially unoccupied, and wherein the at least one shape memory material and portions of the conduit having the at least one shape memory material disposed therein do not extend into the fluid reservoir.

19. A system comprising:
the fluid collection assembly of claim 2;
a fluid storage container configured to hold a fluid; and
a vacuum source fluidly coupled to one or more of the fluid storage container or the fluid collection assembly via the conduit, the vacuum source configured to remove fluid from the fluid collection assembly via the conduit.

20. A method to collect one or more bodily fluids, the method comprising:
positioning an opening of a fluid collection assembly adjacent to a female urethral opening, the fluid collection assembly including:
a fluid impermeable barrier including a chamber at least partially therein, at least one opening configured to be positioned adjacent to a female urethral opening, and a fluid outlet;
at least one porous material disposed in the chamber;
a conduit extending from the fluid outlet into the chamber, the conduit including one or more walls that define a passageway; and
at least one shape memory material at least partially disposed within the one or more walls of the conduit, the at least one shape memory material being sized, shaped, and positioned in the one or more walls to retain a selected geometric configuration, the at least one shape memory material including at least a first shape memory material, the first shape memory material disposed in a portion of the conduit that is opposite the at least one opening;
shaping the fluid collection assembly into the selected geometric configuration, wherein the selected geometric configuration is complementary to contours of anatomy of a patient in a region proximate to the female urethral opening; and
receiving the one or more bodily fluids from the female urethral opening into a chamber of the fluid collection assembly.

21. A fluid collection assembly, comprising:
a fluid impermeable barrier including a chamber at least partially therein, at least one opening configured to be positioned adjacent to a female urethral opening, and a fluid outlet;
at least one porous material disposed in the chamber;
a conduit extending from the fluid outlet into the chamber, the conduit including one or more walls that define a passageway, the conduit including an outer surface, an entirety of the outer surface of the conduit exhibiting a circular cross-sectional shape; and
at least one shape memory material completely disposed within the one or more walls of the conduit, the at least one shape memory material being sized, shaped, and positioned in the one or more walls to retain a selected geometric configuration, the at least one shape memory material includes at least a first shape memory material extending substantially parallel to the longitudinal axis of the conduit, the first shape memory material disposed in a portion of the conduit that is opposite the at least one opening, the first shape memory material including two terminal ends longitudinally spaced from each other, each of the two terminal ends spaced from adjacent terminal ends of the conduit; and
wherein the fluid impermeable barrier and the at least one porous material define a fluid reservoir that is substantially unoccupied, and wherein the at least one shape memory material and portions of the conduit having the at least one shape memory material disposed therein do not extend into the fluid reservoir.
